(19) United States
(12) Patent Application Publication
(10) Pub. No.: US 2021/0238248 A1
(43) Pub. Date: Aug. 5, 2021

(54) Engineering Extracellular Vesicles for Affinity Purification

(71) Applicant: Evox Therapeutics Ltd, Oxford (GB)

(72) Inventors: Virginia Castilla LLORENTE, Oxford (GB); Per LUNDIN, Oxford (GB); Justin HEAN, Oxford (GB); Harsh AMIN, Abingdon (GB)

(21) Appl. No.: 16/972,741

(22) PCT Filed: Jun. 11, 2019
(86) PCT No.: PCT/EP2019/065116

§ 371 Date: Dec. 7, 2020

(30) Foreign Application Priority Data
Jun. 12, 2018 (GB) 1809622.2

(51) Int. Cl. C07K 14/705; C12N 15/88; C12N 15/11

(52) U.S. Cl. CPC C07K 14/705; C12N 15/88; C12N 2310/14; C12N 2320/32; C12N 2310/20; C12N 15/111

(57) ABSTRACT

The present invention pertains to affinity-based isolation and purification of drug-loaded extracellular vesicles, notably exosomes, wherein the exosomes are engineered to enable affinity purification.

### TECHNICAL FIELD

The present invention pertains to affinity-based isolation and purification of drug-loaded extracellular vesicles (EVs), notably exosomes, wherein the EVs are engineered to enable affinity purification.

### BACKGROUND ART

Extracellular vesicles (EVs) are nano-sized vesicles (generally less than 1000 nm in hydrodynamic diameter) that are released by EV-producing cells into the extracellular environment. EVs and in particular exosomes (which are often defined by different parameters, e.g. the presence of various tetraspanins in their membrane or their size) have been shown to be able to transport protein biologics, such as antibodies and siRNAs, into target cells, enabling an entirely novel form of advanced biological therapeutics harnessing the properties of EVs in combination with the specificity of recombinant proteins.

Conventional low-scale methods to prepare and isolate EVs (e.g. exosomes) involve a series of differential centrifugation steps to separate the vesicles from cells or cell debris present in the culture medium into which the EVs are released. Typically, series of centrifugations at e.g. 3,000 g, 10,000 g and 70,000 g or 100,000 g are applied, upon which the resulting pellet at the bottom of the tube is resuspended to a fraction of its original volume with a saline solution to constitute a concentrated EV or exosome solution. However, these methods are fundamentally unsuitable for clinical applications for a number of reasons: (1) the extended length of time needed for the entire process, (2) issues around scale-up, operability in and compliance with a GMP environment, (3) significant risk of contamination by cell debris, (4) poor reproducibility due to operator variability, (5) aggregation of EVs/exosomes resulting from pelleting of the vesicles, (6) low recovery at end of processing, and (7) negative impact on vesicle morphology and thereby potentially biodistribution and activity. There is therefore a need for improved methods of purifying EVs and allow for production of vesicle preparations of therapeutic quality. To that end, PCT application WO2000/044389 discloses methods for preparing membrane vesicles from biological samples through chromatographic techniques, such as anion exchange chromatography and/or gel permeation chromatography. WO2014/168548 discloses a significantly improved isolation and purification method for EVs, namely the use of sequential combinations of filtration and various forms of liquid chromatography, for instance a combination of ultrafiltration and size-exclusion liquid chromatography. However, these technologies are primarily utilizing the physico-chemical properties of the EVs, e.g. their size and charge, regardless of the presence of particular moieties or domains engineered into or onto the EVs and/or the presence of drug cargo in the EVs. Thus, there is a considerable need in the art for improved, specific, and drug-focused methods for purification of EVs for e.g. therapeutic application, in particular for the purification and enrichment of EVs loaded with a particular drug cargo.

### SUMMARY OF THE INVENTION

It is hence an object of the present invention to overcome the above-identified problems associated with the isolation and purification of EVs, and in particular EVs from EV populations loaded with various types of drug cargo. Furthermore, the present invention aims to satisfy other existing needs within the art, for instance to develop generally applicable affinity purification strategies for EV purification at high yields and with high specificity.

The present invention achieves these and other objectives by inventive protein engineering which enables both drug loading into EVs and affinity-based purification of such EVs, using a single fusion protein construct. In a first aspect, the present invention relates to such inventive fusion proteins, which comprise at least the following components: (i) an EV polypeptide (also interchangeably termed exosomal polypeptide, EV protein, exosomal protein, and similar terms), (ii) a purification moiety, and (iii) a drug loading moiety. In a second aspect, the present invention relates to a complex between the fusion protein and a drug of interest (DOI). The interaction between the fusion protein and the DOI is typically based on non-covalent interaction(s) between the drug-loading moiety of the fusion protein and a site, region, domain, or structural or chemical feature of the DOI.

In a highly inventive aspect, the present invention relates to an extracellular vesicle (EV) comprising the fusion protein of the present invention. Furthermore, as abovementioned, the fusion protein is capable of interacting with a DOI with the aid of its drug-loading moiety and thus the EV may further comprise a DOI, which may be present in the form of a complex with the fusion protein, but which may also have been released or dissociated from the fusion protein and exist as a free DOI in the EV. Preferred DOIs as per the present invention include proteins, peptides, and/or nucleic acid (NA)-based agents, for instance various forms of RNA therapeutics such as mRNA, miRNA, short hairpin RNA, guide RNA, single guide RNA, or combinations thereof, e.g. a Cas-CRISPR ribonucleoprotein. The EVs of the present invention are preferably exosomes or microvesicles, or any other type of EVs obtainable from the endo-lysosomal pathway or the plasma membrane.

In further aspects, the present invention relates to polynucleotides encoding for the fusion proteins herein, as well as vectors and cells comprising such polynucleotides. The cells of the present invention may further comprise said fusion proteins as well as polynucleotides encoding for a DOI.

In a further important aspect, the present invention relates to methods for producing EVs comprising a fusion protein as per the present invention. These methods typically comprise the steps providing a population of EVs comprising a fusion protein as per the present invention and exposing such a population to an affinity purification system, for instance a chromatography system allowing for binding of the purification domain comprised in the EVs to a ligand on a solid phase, allowing for capturing of the EVs comprising the fusion protein and normally a DOI. More specifically, such methods typically comprise the steps of (i) introducing into an EV-producing cell a polynucleotide which encodes for the fusion protein in question, and (ii) allowing for the EV-producing cell to produce EVs comprising the fusion protein. In addition, a polynucleotide encoding for a drug of interest (DOI) may be introduced into the EV-producing cell, in order to allow for the EV-producing cell to produce EVs comprising the fusion protein, wherein the drug-loading moiety of the fusion protein binds to the DOI and transports it into the EV, thus creating EVs comprising both the fusion protein and the DOI, typically initially as part of a complex but as a result of release of the DOI from the fusion protein, as single entities. The polynucleotide which encodes for a DOI may encode for various types of drug cargo, including proteins, peptides, mRNAs, short hairpin RNAs, miRNAs, pri-miRNAs, pre-miRNAs, antisense oligonucleotides, guide RNAs, single guide RNAs, circular RNAs, piRNAs, tRNAs, rRNAs, snRNAs, IncRNAs, ribozymes, DNAs, and/or any combination or derivative thereof. Chemically modified oligonucleotide cargos are particularly preferable.

In yet another aspect, the present invention relates to a method of purification of EVs comprising a DOI. The method typically comprises the steps of (i) providing an EV according to the invention, (ii) allowing the purification moiety of the fusion protein comprised in the EV to bind a purification ligand; and (iii) removing EVs that have not bound to the purification ligand.

In further aspects, the present invention relates to a pharmaceutical composition comprising the EVs and a pharmaceutically acceptable carrier, as well as medical uses and medical treatments using the pharmaceutical composition or the EVs (and populations of EVs).

### BRIEF DESCRIPTION OF THE FIGURES

FIG. 1 shows a schematic illustration of an EV, such as an exosome, comprising a fusion protein which comprises a drug-loading moiety, an exosomal protein, and a purification domain. The EV furthermore comprises a drug cargo molecule, in this case as a non-limiting example an RNA cargo molecule in the form of a hairpin RNA, which is bound by the drug-loading moiety and shuttled into the EV that is subsequently purified using the purification domain of the fusion protein.

FIG. 2 compares the output from an EV batch purified using two different purification processes. The EVs were genetically engineered to comprise a fusion protein comprising CD63 as the exosome protein, a ZZ domain as the purification domain, and Cas6 as a drug-loading moiety for mRNA drug loading. Nanoluciferase mRNA was loaded into the exosomes with the aid of the Cas6 mRNA-loading domain and the total mRNA translated after exosome-mediated delivery was assessed using a bioluminescence readout. As can be seen in FIG. 2, a combination of TFF and bead-elute size exclusion chromatography (BE-SEC purification) yielded an RLU of approximately 5E8, whereas an affinity chromatography process based on an IgG column resulted in an RLU of approximately 3.2E9, as a result of higher enrichment of the Nanoluciferase mRNA in the final EV population.

FIG. 3 shows a schematic illustration of an EV comprising a fusion protein which comprises a drug-loading moiety, an exosomal protein, a purification domain, and inserted between the purification domain and the exosomal protein a cleavage domain for enzymatic cleavage. This design enables affinity-based capture using the purification domain followed by simultaneous removal of the purification domain and elution of the drug-loaded EVs. An shRNA is used to exemplify the drug cargo molecule but this approach is equally applicable to other forms of drug cargo such as sgRNA, antisense oligonucleotides, miRNA, mRNA, proteins, peptides, etc.

FIG. 4 shows the percentage of single guide RNA (sgRNA) positive EVs in a final EV population post purification using either (i) TFF and BE-SEC (BE-SEC purification), (ii) MBP-based affinity purification (MBP affinity purification), or (iii) MBP-based purification using a fusion protein further comprising a TEV cleavage domain for enzymatic removal of the MBP domain (MBP-TEV affinity purification). The MBP-based affinity capture (with our without the TEV cleavage linker) resulted in IGF2BP1 sgRNA being present in approximately 90% of all EVs post purification, whereas the BE-SEC purified EVs were approximately 40% IGF2BP1 sgRNA positive (FIG. 4).

### DETAILED DESCRIPTION OF THE INVENTION

The present invention relates to inventive fusion proteins and related aspects and embodiments, which enable production and affinity purification of drug-loaded EVs, in particular exosomes.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention.

In a first aspect, the present invention relates to a fusion protein which comprises at least the following components: (i) an EV polypeptide, (ii) a purification moiety (interchangeably termed a “purification domain”), and (iii) a drug loading moiety. In one important embodiment, the drug-loading moiety is a nucleic acid (NA)-binding protein or a protein-binding domain, which enable loading into the EVs of NAs (for instance RNA agents) or proteins (for instance protein-based drugs such as enzymes, tumor suppressors, or antibodies). The fusion proteins of the present invention thus have three functionalities, i.e. (1) the EV polypeptide drives transportation into EVs of the entire fusion protein, (2) the drug-loading moiety enables the fusion protein to carry with it one or more drug of interest (DOI) into or onto the EV, and (3) the purification moiety enables purification and isolation of only the EVs that contain the drug of interest. This tri-functional construct thereby solves a significant challenge in the EV field, i.e. the isolation of pure EV populations which are enriched in a DOI, meaning that the final drug substance and drug product will contain fewer (if any) empty, non-drug loaded EVs.

The EV polypeptides which drive loading of the fusion proteins and thus the DOI into EVs may be selected from the group comprising the following non-limiting examples: CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain, CD133, CD138 (syndecan-1), CD235a, ALIX, Syntenin-1, Syntenin-2, Lamp2a, Lamp2b, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, ARRDC1, LFA-1, LGALS3BP, Mac-1 alpha, Mac-1 beta, MFGE8, PTGFRN, SLIT2, STX3, TCRA, TCRB, TCRD, TCRG, TSG101, VTI1A, VTI1B, Fibronectin, Rab7, 14-3-3 zeta/delta, 14-3-3 epsilon, HSC70, HSP90, HSPA13 and any other EV polypeptides, and any combinations, derivatives, fragments, domains, or regions thereof.

In preferred embodiments, the EV polypeptide is a transmembrane or membrane-associated polypeptide. The use of a transmembrane exosome protein makes it possible to configure the drug-loading moiety on the luminal side and the purification moiety on the extravesicular side of the EV membrane. This configuration supports loading of e.g. sensitive RNA-based drugs such as mRNA or shRNA or other sensitive therapeutic agents (e.g. a Cas9 nuclease) into the EV where they are protected from degradation, while exposing the purification tag on the outside to enable access and binding to an affinity purification ligand or moiety. Suitable transmembrane or membrane-associated EV polypeptides may be selected from the group comprising CD63, CD81, CD9, CD82, CD44, CD47, CD55, LAMP2B, ICAMs, integrins, ARRDC1, annexin, and any other EV polypeptides, and any combinations, derivatives, domains, or regions thereof. A non-limiting example could be a protein such as CD63, which has 4 transmembrane domains and both the N terminus and the C terminus of the protein present on the inside of EVs. Thus, one or more drug-loading moieties (for instance an RNA-binding protein) may be fused onto the N terminus and/or the C terminus, whereas a purification moiety may be introduced into one or more of the extravesicular regions, such as for instance in the 1^{st} and/or the 2^{nd} loops. In one particularly preferred embodiment, the fusion protein can be schematically described as follows (N terminally to C terminally):
CD63—ZZ domain inserted into the 1^{st} and 2^{nd} loop of CD63—RNA binding protein
   In this particular embodiment, the RNA-binding protein may for instance be Cas6, Cas9, PUF, TAR RNA-binding protein (TRBP), or any other type of RNA-binding protein or other type of drug-loading protein (such as a region or domain or polypeptide mediating e.g. a protein-protein interaction). Alternatively, it is also possible to engineer both the drug-loading moiety and the purification tag into the extravesicular parts of a fusion protein in question, for instance CD63 or CD81 or CD9, etc. The structure of such a fusion construct could be introduction of a purification tag in the 1^{st} or 2^{nd} loop of the protein and introduction of a drug-loading moiety in the 2^{nd} or 3^{rd} loop of the protein, or vice versa.

In other similar embodiments, exosomal proteins having a single transmembrane region may be utilized. Suitable examples of such transmembrane exosomal proteins include CD63, CD47, Lamp2b, ARRDC1, L1CAM, etc. In the cases of such transmembrane exosomal proteins, the drug-loading moiety is advantageously fused onto the luminal terminal of the protein and the purification moiety is fused to the extravesicular terminal of the protein.

In alternative embodiments, the EV polypeptide may be a non-transmembrane polypeptide which is fused to a transmembrane polypeptide which locates the fusion protein to the exosomal membrane. Suitable examples of such non-transmembrane EV polypeptides include ALIX, syntenin-1, syntenin-2, Lamp2b, syndecan-1, syndecan-2, syndecan-3, syndecan-4, and any other non-transmembrane exosome proteins. These non-transmembrane exosome proteins may advantageously be fused with e.g. the transmembrane domain of cytokine receptors, co-receptors, or signal transducers (e.g. the TNF receptor 1 or 2 or gp130 or IL23R or IL17R, etc.), to enable anchoring them into the EV membrane. Similarly, it is also possible to fuse a non-transmembrane EV polypeptide with a transmembrane EV polypeptide, thereby further enhancing the exosome transport of the fusion protein.

In one preferred embodiment of the present invention, the NA-binding protein of the fusion protein is selected from the group comprising mRNA-binding proteins, miRNA-binding proteins, pre-rRNA-binding proteins, tRNA-binding proteins, small nuclear or nucleolar RNA-binding proteins, non-coding RNA-binding proteins, transcription factors, nucleases, RISC proteins, and any combination, derivative, domain, region, site or part thereof that can fulfil the same purpose, namely to bind to an NA molecule of interest.

Particularly suitable NA-binding protein may be selected from any one of PUF, PUF531, PUFx2 (i.e. a fusion protein comprising at least two RNA-binding domains of a PUF protein), DDX3X, EEF2, EEF1A1, HNRNPK, HNRNPM, HNRNPA2B1, HNRNHPH1, HNRNPD, HNRNPU, HNRNPUL1, NSUN2, Cas6, Cas13, Cas9, WDR1, HSPA8, HSP90AB1, MVP, PCB1, MOCS3, DARS, ELC2, EPRS, GNB2L1, IARS, NCL, RARS, RPL12, RPS18, RPS3, RUVBL1, TUFM, hnRNPA1, hnRNPA2B1, DDX4, ADAD1, DAZL, ELAVL4, ELAVL1, IGF2BP3, HNRNPQ, RBFOX1, RBFOX2, U1A, PPR family, ZRANB2, NUSA, IGF2BP1, IGF2BP2, IGF2BP3, Lin28, KSRP, SAMD4A, TDP43, FUS, FMR1, FXR1, FXR2, EIF4A1-3, MS2 coat protein, as well as any domains, parts or derivates, thereof.

More broadly, particular subclasses of RNA-binding proteins and domains may be used as the drug-loading moiety, e.g. mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins and transcription factors (TFs). Furthermore, various domains and derivatives may be used as the NA-binding domain to transport an NA cargo into EVs. Examples include DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, Ribosomal_L7Ae and any combination, derivative, domain, region, site, mutated variants or part(s) thereof. Such RNA-binding domains may be present in a plurality, alone or in combination with others, and may also form part of a larger RNA-binding protein construct as such, as long as their key function (i.e. the ability to transport an NA cargo of interest, e.g. an mRNA or a short RNA) is maintained.

The NA-binding domains of the present invention have been selected to allow for programmable, modifiable affinity between the NA-binding domain and the NA cargo molecule, enabling production of EVs comprising fusion polypeptides comprising the NA-binding domain and at least one NA cargo molecule, wherein the NA-binding domain of the fusion polypeptide construct interacts in a programmable, reversible, modifiable fashion with the NA cargo molecule, allowing for both loading into EVs and release of the NA cargo molecule either in EVs and/or in or in connection with target cells. This will be an advantage for instance when the NA cargo molecule is intended to be released in a recipient cell. The defined association time can then be modulated to release the RNA molecule when needed, but not in the producer cell. This is particularly important for the present invention so as to ensure that the cargo nucleic acid is stably associated with the EV whilst the purifcation process occurs so that the purified EV retains its cargo but also that the cargo nucleic acid is releasable and therefore functional (bioactive) when delivered to the target/recipient cell.

Thus, in advantageous embodiments, the present invention relates to eukaryotic NA-binding proteins fused to exosomal proteins. In a preferred embodiment, the NA-binding domain(s) is(are) from the PUF family of proteins, for instance PUF531, PUFengineered, and/or PUFx2. Importantly, PUF proteins are preferably used in the EV-mediated delivery of mRNA due to the sequence-specificity of the PUF proteins which enables highly controlled and specific loading of the mRNA drug cargo. Advantageous fusion protein constructs include the following non-limiting examples: CD63-PUF531, CD63-PUFx2, CD63-PUFengineered, CD81-PUF531, CD81-PUFx2, CD81-PUFengineered, CD9-PUF531, CD9-PUx2, CD9-PUFengineered, and other transmembrane-based fusion proteins, preferably based on tetraspanin exosomal proteins fused to one, two or more PUF proteins. Advantageous fusion proteins comprising PUF proteins and at least one soluble exosomal protein include the following non-limiting examples: syntenin-PUF531, syntenin-PUx2, syntenin-PUFengineered, syndecan-PUF531, syndecan-PUx2, syndecan-PUFengineered, Alix-PUF531, Alix-PUx2, Alix-PUFengineered, as well as any other soluble exosome protein fused to a PUF protein.

Similarly, the RNA sequence that Cas6 can recognize can be engineered to be inserted into an NA molecule of interest. Cas13 can be engineered to only bind its defined RNA target and not degrade it. By changing the sequence of the sgRNA molecule the Cas13-sgRNA complex can be modulated to bind any RNA sequence between 20-30 nucleotides. As with the PUF-based NA-binding domains, the Cas proteins represent a releasable, reversible NA-binding domain with programmable, modifiable sequence specificity for the target NA cargo molecule, enabling higher specificity at a lower total affinity, thereby allowing for both loading of the NA cargo into EVs and release of the NA cargo in a target location. Such PUF and Cas nucleic acid binding domains are particularly suited to loading of mRNA into EVs for purification by the present invention.

Embodiments employing PUF proteins and CRISPR-associated polypeptides (Cas), specifically Cas6 and Cas13, and/or various types of NA-binding aptamers in the invention have the advantageous effect, as mentioned above, of achieving a programmable, lower affinity interaction between the NA-binding domain and the NA cargo molecules. This enables the present invention to efficiently load EVs in EV-producing cells, whilst also enabling release of NA cargo in suitable locations (typically inside a target cell) where the lower affinity and the releasable nature of the interaction between the NA cargo molecule and the NA-binding domain is highly advantageous. In detail, the present invention allows for sequence-specific low-affinity or medium-affinity binding to stretches of nucleotides that are longer and thereby more specific, for instance 6 nt in length, or 8 nt in length. The longer length of binding site enables a range of different mutations to be introduced which generate binding sites with a range of modified binding affinities, thus producing the programmable lower affinity interactions mentioned above. For instance, introduction of a single point mutation into a 6 or 8 nucleotide region will subtly modify the binding affinity, providing more scope to introduce one or more mutations which affect the binding affinity of the protein for the nucleic acid. Similarly, requiring a longer stretch of nucleotides to be bound results in a larger number of amino acids which are capable of interacting with the longer nucleotide sequence and thus providing more possibilities for mutating those interacting amino acids and again producing a larger range of possible protein mutants with a variety of binding affinities. Both the longer nucleotide binding site and the larger protein binding sites of PUF, Cas6 and Cas13 provide advantages in enabling a wide range of affinities to be achieved by mutation.

Thus, this longer sequence affords greater possibilities to engineer the nucleic acid and/or the binding protein to tailor the binding affinity specifically to an individual cargo of interest if needed to improve the release of that cargo nucleic acid. The ability to control the affinity of binding to the nucleotide cargo and thus modify and control the releasability of the nucleotide cargo is a significant advantage of the present invention, resulting in sucessful purification of NA loaded EVs and subsequent delivery and release of those nucleic acids in a bioactive state (unbound). This is particularly important for mRNA which is only bioactive when released from the NA-binding domain so that it can then be actively translated. In addition to loading into EVs of NA-based drug cargo molecules, the present invention is highly suitable for loading of amino acid-based drug molecules. Loading of polypeptide-based drugs can often advantageously be achieved by using a single fusion protein between a polypeptide-based DOI and an exosome protein, but the typically non-covalent interaction between (i) a drug-loading moiety of the fusion proteins of the present invention and (ii) a DOI in the form of a peptide and/or protein may be a preferred EV loading method in the case of e.g. soluble therapeutic protein and/or peptide DOIs. Non-limiting examples of drug-loading moieties of the present invention which are advantageous to use in the context of loading of amino acid-based DOI(s) include: importin as a means for loading a DOI comprising a nuclear localization signal (to which the importin protein binds); the CRY2-CIBN interaction under blue light as a means for loading a DOI, whereby a fusion between CRY2 and an exosomal protein and CIBN and the DOI (or vice versa) allows for trafficking to the exosome; any pH dependent interaction between a DOI and it's binding motif which is grafted onto the fusion protein of the present invention; an Fc-binding polypeptide used in this particular embodiment as a drug-loading domain, whereby it enables binding to and loading of polypeptides with a Fc domain, for instance antibodies or proteins artificially engineered to comprise an Fc domain; the Spy-Catcher Spy-Tag system, whereby one of either the Tag or the Catcher is fused to the DOI and its binding partner is fused to the fusion protein: and/or the SnoopTag-SnoopCatcher system.

Importantly, the inventive design of the fusion proteins of the present invention enables affinity-based purification of only the EVs that contain the drug-loading moiety and therefore the DOI. Suitable purification moieties of the present invention include the following non-limiting examples: a receptor, an antibody-binding polypeptide, an Fc-binding polypeptide, poly-histidine, glutathione S-transferase (GST), maltose-binding protein (MBP), calmodulin-binding peptide (CBP), intein-chitin binding domain (I-CBD), streptavidin, avidin, FLAG epitope tag, HA epitope tag, T7tag, S-tag, CLIP, DHFR, cellulose-binding domain, and any combination, derivative, domain or part thereof.

In one particular advantageous embodiment, the Fc-binding polypeptide is selected from the group comprising Protein A, Protein G, Protein A/G, Protein L, Protein LG, Z domain, ZZ domain, human FCGRI, human FCGR2A, human FCGR2B, human FCGR2C, human FCGR3A, human FCGR3B, human FCGRB, human FCAMR, human FCERA, human FCAR, mouse FCGRI, mouse FCGRIIB, mouse FCGRIII, mouse FCGRIV, mouse FCGRn, FcIII peptide, and any combination, derivative, domain or part thereof.

As is normally the case with fusion proteins, the three components that are normally included in the fusion protein (i.e the drug-loading moiety, the exosomal protein, and the purification domain) may be linked directly in a contiguous fashion in the fusion protein, or they may be linked and/or attached to each other using a variety of linkers, release domains or release sites, cleavage sites or cleavage domains. For instance, in certain embodiments when it is desirable to remove the purification domain, a cleavage linker may be introduced between the exosomal protein and the purification domain, thus enabling cleavage (for instance through enzymatic activity) of the cleavage linker and thereby removal of the purification domain. Suitable examples of such cleavage linkers for purification domain removal are TEV or SUMO linkers, which can be cleaved by two different types of proteases, respectively. Another advantageous modification of the fusion protein entails inserting a release domain between the drug-loading moiety and the exosomal protein, to enable release of the DOI once it has been loaded into an EV, such as an exosome. A suitable example of such a drug release linker is an intein.

In a further aspect, the present invention relates to a complex between a fusion protein as described herein and a DOI. This complex can be seen as a system of two components, namely the fusion protein and the drug of interest (DOI). Typically, the interaction between the two components of the complex (i.e. the system) is based on non-covalent interactions between the drug-loading moiety and the DOI. The DOI included in such complexes may for instance be one or more NA agents, one or more proteins, and/or one or more peptides, or also any combination thereof, for instance a ribonucleoprotein complex such as e.g. Cas9-sgRNA. In the case of NA agents, such NA agents may be selected from the group comprising the following non-limiting examples: single-stranded RNA or DNA, double-stranded RNA or DNA, oligonucleotides such as siRNA, splice-switching RNA, pri-miRNA, pre-miRNA, circular RNA, piRNA, tRNA, rRNA, snRNA, IncRNA, CRISPR guide strands (gRNA, sgRNA), short hairpin RNA (shRNA), miRNA, cyclic dinucleotides, antisense oligonucleotides, ribozyme, polynucleotides such as mRNA, mini-circle DNA, plasmids, plasmid DNA, or any other RNA or DNA vector. Of particular interest are nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, DNA mixmers, etc.

In advantageous embodiments, the NA may comprise either at least one naturally occurring and/or at least one artificially introduced region, domain, component, or site to which the NA-binding protein binds. Such a region, structure, domain, site or sequence may be present naturally in the NA agent or may be introduced into the NA agent through e.g. genetic engineering. Non-limiting examples of naturally occurring NA regions, structures or sites to which an NA-binding protein could bind are the hairpin structure of e.g. an shRNA or a particular sequence of nucleotides. Artificially introduced region, structure, domain, functionality, site or sequence may be essentially any such feature to which an NA-binding protein can bind and attach, for instance particular nucleotide sequences, particular nucleotide modifications, stem loops, hairpins, blunt ends, as well as various other features capable of being introduced into NA agents, and in particular RNA agents. In one advantageous embodiment, the NA agent may encode for a therapeutic protein, e.g. the NA agent may be a messenger RNA (mRNA) encoding for a protein of interest. The therapeutic protein may be of essentially any type or nature. Non-limiting examples of proteins of interest (Pols) that may be encoded for by the NA (in this embodiment advantageously an mRNA) cargo molecule include the following: antibodies, intrabodies, single chain variable fragments (scFv), affibodies, bi- and multispecific antibodies or binders, receptors, ligands, enzymes for e.g. enzyme replacement therapy or gene editing, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In one preferred embodiment, the encoded protein is a CRISPR-associated (Cas) polypeptide with intact nuclease activity which is associated with (i.e. carries with it) an RNA strand that enables the Cas polypeptide to carry out its nuclease activity in a target cell once delivered by the EV. Alternatively, in another preferred embodiment, the Cas polypeptide may be catalytically inactive, to enable targeted genetic engineering. Yet another alternative may be any other type of CRISPR effector such as the single RNA guided endonuclease Cpf1. The inclusion of Cpf1 is a particular preferred embodiment of the present invention, as it cleaves target DNA via a staggered double-stranded break, Cpf1 may be obtained from species such as, Acidaminococcus or Lachnospiraceae. In yet another exemplary embodiment, the Cas polypeptide may also be fused to a transcriptional activator (such as the P3330 core protein), to specifically induce gene expression. Additional preferred embodiments include proteins selected from the group comprising enzymes for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN -acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other preferred embodiments, the Pol may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The Pols are typically proteins or peptides of human origin unless indicated otherwise by their name, any other nomenclature, or as known to a person skilled in the art, and they can be found in various publicly available databases such as Uniprot, RCSB, etc.

Naturally, all types of proteins encoded for by an NA agent as per the present invention may be modified, engineered, truncated, derivatized, and fused to various fusion partners. Particularly advantageous proteins encoded for by the NA agent (typically in the form of an mRNA) include: lysosomal enzymes or transporters such as NPC1, NPC2, GBA, GLA, cystinosin, Lamp2, Limp2 and acetylhexosaminidase; urea cycle enzymes such as ASS, ASL and ARG1; structural proteins such as dystrophin, mini-dystrophin, micro-dystrophin, utrophin, fibrillin, etc.; enzymes and proteins deficient in inherited errors of metabolism (IEMs), antibodies, intrabodies, antibody domains, antibody derivatives, single-chain antibodies, single-domain antibodies, scFvs, tumor suppressors, transcription factors, nucleases such as Cas9, Cpf1, Cas6, TALENS, TALES, and zinc fingers, etc. As will be clear to the skilled person, the list of proteins that could be loaded and delivered with the aid of the inventive EV design as per the present invention are essentially unlimited and the above merely serves as non-limiting examples of proteins particularly suitable to be delivered as mRNA cargo molecules.

In a further aspect, the present invention relates to EVs comprising the inventive fusion proteins herein. Importantly, in certain embodiments, EVs may be present as single vesicles but EVs are normally present in substantial plurality, i.e. in populations comprising anywhere from thousands to millions to billions to trillions and even more EVs. In advantageous embodiments, the EVs comprise the fusion protein, wherein the fusion protein is present in the EVs in the form of a complex with a DOI, such as an RNA molecule or a protein. In certain embodiments, the DOI may be transported into the EVs with the aid of the drug-loading moiety followed by dissociation of the DOI from the complex in the EV and/or in a target environment, for instance inside or outside a target cell. Thus, said complex between the DOI and the fusion protein may be long-lasting or relatively transient, depending on the design, nature and activity of the DOI and the fusion protein. However, the key success criteria for such a complex between the fusion protein and the DOI is that the DOI is efficiently loaded into EVs, that affinity purification is enabled as a result of the presence of the purification domain, and that the DOI is capable of exerting its activity, e.g. translation of an mRNA DOI into a protein or binding of an antibody DOI to its target antigen.

The terms “extracellular vesicle” or “EV” or “exosome” or “genetically modified/genetically engineered exosome” or “modified exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endosomal, lysosomal and/or endo-lysosomal pathway), an apoptotic body, ARMMs (arrestin domain containing protein 1 [ARRDC1]-mediated microvesicles), a microparticle and vesicular structures, etc. The terms “genetically modified” and “genetically engineered” EV indicates that the EV is derived from a genetically modified/engineered cell usually comprising a recombinant or exogenous NA and/or protein product which is incorporated into the EVs produced by those cells. The term “modified EV” indicates that the vesicle has been modified either using genetic or chemical approaches, for instance via genetic engineering of the EV-producing cell or via e.g. chemical conjugation, for instance to attach moieties to the exosome surface. The sizes of EVs may vary considerably but an EV typically has a nano-sized hydrodynamic diameter, i.e. a diameter below 1000 nm. Clearly, EVs may be derived from any cell type, in vivo, ex vivo, and in vitro. Preferred EVs include exosomes and microvesicles, but other EVs may also be advantageous in various circumstances. Furthermore, said terms shall also be understood to relate to extracellular vesicle mimics, cell membrane-based vesicles obtained through for instance membrane extrusion, sonication, or other techniques, etc. Furthermore, when teachings herein refer to EVs in singular and/or to EVs as discrete natural nanoparticle-like vesicles it should be understood that all such teachings are equally relevant for and applicable to a plurality of EVs and populations of EVs.

As abovementioned, the purification moiety of the fusion protein is preferably present at least partially on the outside of the EV, to enable e.g. affinity-based purification of the EVs which are engineered to be loaded with a DOI. Typically, the purification moiety of the fusion protein designed to be able to interact with a purification ligand, purification matrix, or purification equipment or machine (interchangeably jointly termed purification binders or purification ligands). Such purification ligands may be specific ligands that interact with high specificity with a particular purification moiety (for instance, an Fc binding polypeptide such as a Z domain interacting with the Fc-portion of an antibody, wherein said antibody is attached to a chromatography matrix), or may be e.g. a His tag which interacts with various metal atoms, for instance nickel atoms.

In a highly preferred embodiment, the EVs as per the present invention are exosomes, microvesicles (MVs), or any other type of vesicle which is secreted from the endosomal, endolysomal and/or lysosomal pathway or from the plasma membrane of a parental cell. Generally speaking, the present invention relates to any type of vesicular structure secreted, produced by, and/or derived from a cell, including but not limited to exosomes, microvesicles, ARRDC1-mediated microvesicles (ARMMs), extruded vesicles, extruded cells and/or cell membranes, various lipid-based vesicles including hybrids between EVs and lipids, etc.

In an additional aspect, the present invention relates to a polynucleotide encoding for the fusion proteins herein. The polynucleotide constructs may be present in various different forms and/or in different vectors. For instance, the polynucleotides may be essentially linear, circular, and/or has any secondary and/or tertiary and/or higher order structure. Furthermore, the present invention also relates to vectors comprising the polynucleotides, e.g. vectors such as plasmids, any circular or linear DNA polynucleotide, mini-circles, viruses (such as adenoviruses, adeno-associated viruses, lentiviruses, retroviruses), mRNAs, and/or modified mRNAs.

In further aspects, the present invention relates to cells comprising (i) at least one polynucleotide construct according to the present invention and/or (ii) at least one fusion protein as per the present invention. Furthermore, the present invention also relates to cells comprising the EVs as per the present invention, i.e. the EVs when they are formed but not yet released from an EV-producing cell. The EV-producing cells may be present in the form of e.g. primary cells, cell lines, cells present in a multicellular organism, or essentially any other type of cell source and EV-producing cell material. The terms “source cell” or “EV source cell” or “parental cell” or “cell source” or “EV-producing cell” or any other similar terminology may be understood to relate to any type of cell that is capable of producing EVs under suitable conditions, for instance in suspension culture or in adherent culture or any in other type of culturing system. Source cells as per the present invention may also include cells producing exosomes in vivo. The source cells per the present invention may be selected from a wide range of cells and cell lines which may grow in suspension or adherent culture or being adapted to suspension growth. The source cells per the present invention may be selected from the group comprising:mesenchymal stem or stromal cells (obtainable from e.g. bone marrow, adipose tissue, Wharton's jelly, perinatal tissue, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, epithelial cells from the airways or alveolae, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. Generally, EVs may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method. When treating neurological diseases, one may contemplate to utilize as source cells e.g. primary neurons, astrocytes, oligodendrocytes, microglia, and neural progenitor cells. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication. For instance, in the context of treating inflammatory or degenerative diseases, allogeneic MSCs or AEs may be highly beneficial as exosome-producing cell sources due to the inherent immuno-modulatory of their EVs and in particular their exosomes. Cell lines of particular interest include human umbilical cord endothelial cells (HUVECs), human embryonic kidney (HEK) cells such as HEK293 cells, HEK293T cells, serum free HEK293 cells, suspension HEK293 cells, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, airway or alveolar epithelial cells, fibroblasts, endothelial cells, epithelial cells, etc.

Furthermore, the cells as per the present invention may normally comprise a polynucleotide encoding for the tri-domain fusion proteins herein as well as another or the same polynucleotide encoding for a DOI. Thus, the source cells may be transfected so as to produce a single, double or multiply stable source cell line. Single stable cell lines are advantageous because production of EVs is simplified by requiring only the transfection of a single construct. For instance, in one embodiment, the DOI may be encoded by a polynucleotide construct inserted into the EV-producing cell. The DOI may for instance in advantageous enbodiments be an NA agent such as an mRNA, a short hairpin RNA, a miRNA, pri-miRNA, pre-miRNA, an antisense oligonucleotide, a guide RNA, a single guide RNA, circular RNA, piRNA, tRNA, rRNA, snRNA, IncRNA, ribozymes, DNA, and/or any combination or derivative thereof. Alternatively, the DOI may be in the form of e.g. a protein and/or a peptide, which is then encoded for by a polynucleotide construct, which may be the same or a different construct to the one encoding for the fusion proteins herein.

In an additional aspect, the present invention relates to methods for producing EVs comprising the fusion protein according to the invention. The methods may typically comprise the steps of (i) introducing into an EV-producing cell a polynucleotide which encodes the fusion protein (i.e., a fusion protein comprising at least an exosomal protein, a purification domain, and a drug-loading moiety); and (ii) allowing for the EV-producing cell to produce EVs comprising the fusion protein. In a further aspect, the present invention also relates to a method for producing EVs comprising a DOI, the method comprising the steps of (i) introducing into an EV-producing cell a polynucleotide encoding for a fusion protein and a polynucleotide encoding for a DOI; and, (ii) allowing for the EV-producing cell to produce EVs comprising the fusion protein and the DOI as encoded for by the polynucleotide, wherein the drug-loading moiety of said fusion protein binds to the DOI and transports it into the EV. As abovementioned, the polynucleotide encoding for the DOI may encode for a DOI which may be, e.g., a protein, a peptide, an mRNA, a short hairpin RNA, a miRNA, pri-miRNA, pre-miRNA, an antisense oligonucleotide, a guide RNA, a single guide RNA, circular RNA, pi RNA, tRNA, rRNA, snRNA, IncRNA, ribozymes, DNA, and/or any combination or derivative thereof.

Preferably the source cells are stably transfected with the construct encoding the fusion protein(s) of the invention, such that a stable cell line is generated. This advantegously results in consistent production of EVs of uniform quality and yield. The EV-producing cells may be genetically modified with the at least one polynucleotide construct using essentially any non-viral or viral method for introducing a polynucleotide into a cell. The polynucleotide encoding for the fusion protein and the polynucleotide encoding for the DOI may be introduced into the EV-producing cell using essentially any non-viral or viral method for introducing a polynucleotide into a cell. Suitable methods for introducing the polynucleotides include transfection using polycations such as PEI, lipid-based transfection reagents such as Lipofectamine (RTM), lentiviral transduction, CRISPR-Cas guided insertion, Flp-In system, transposon system, electroporation, DEAE-Dextran transfection, and calcium phosphate transfection. The choice of method for introducing the polynucleotide into an EV-producing cell will depend on various parameters, including choice of cell source, the nature and characteristics of the polynucleotide vector (e.g. if the vector is a plasmid or a minicircle or e.g. a linear DNA polynucleotide or an mRNA), and the level of compliance and control needed. Similarly, immortalization of EV-producing cells to create stable cell lines can be achieved using techniques that are well known in the art of cell line development, including hTERT-mediated immortalization, transcription factor immortalization, E1/E2 immortalization or other virus-mediated immortalization techniques, etc.

In a further aspect, the present invention relates a method of purification of EVs comprising a DOI. The method typically comprises the steps of (i) providing an EV comprising the fusion proteins as per the present invention, (ii) allowing the purification domain of the fusion protein comprised in the EV to bind a purification ligand to enable isolation and/or purification of the EVs, and (iii) removing EVs that have not bound to the purification ligand.

In a preferred embodiment, the purification ligand is attached to a solid phase, to enable e.g. chromatography and/or membrane-based purification. Affinity chromatography is generally based on the highly selective interaction between an immobilized ligand and a structural element on the target biomolecule, in this case on the target EV or exosome. The high selectivity of affinity chromatography may be provided by multiple molecular interactions (including hydrogen bonds, hydrophobic interactions, ionic interactions and/or van der Waals interactions) between the purification ligand on e.g. the chromatography matrix and the purification domain forming part of the fusion proteins comprised in the EVs of the present invention. Suitable purification domains for affinity-based purification of the fusion protein-containing EVs include receptors, antibody-binding polypeptides, Fc-binding polypeptides, poly-histidin, glutathione S-transferases (GST), maltose-binding proteins (MBP), calmodulin-binding peptides (CBP), intein-chitin binding domains (I-CBD), streptavidins, avidins, FLAG epitope tags, HA epitope tags, T7 tags, S-tags, CLIP tags, DHFR, cellulose-binding domains, and any combination, derivative, domain or part thereof. In a particularly advantageous embodiment, the purification domain is an Fc-binding polypeptide, which may be selected from the group comprising Protein A, Protein G, Protein A/G, Protein L, Protein LG, Z domain, ZZ domain, human FCGRI, human FCGR2A, human FCGR2B, human FCGR2C, human FCGR3A, human FCGR3B, human FCGRB, human FCAMR, human FCERA, human FCAR, mouse FCGRI, mouse FCGRIIB, mouse FCGRIII, mouse FCGRIV, mouse FCGRn, FcIII peptide, and any combination, derivative, domain or part thereof. As abovementioned, in addition to the purification domain, the fusion proteins of the present invention may include cleavage sites, which enables removal of the purification domain itself post capturing by a purification ligand on e.g. an affinity chromatography column. After such a cleavage reaction, the enzyme catalyzing the cleavage may be removed using e.g. size-exclusion chromatography (using for instance Captocore 700 resin, to allow for bead-elute chromatography) or charged membrane/ion exchange chromatography (for instance using Sartobind Q or Mustang Q ion exchangers).

Generally speaking, the affinity purification of the engineered EVs of the present invention will result in a very pure, highly drug-enriched EV population. However, additional isolation, purification, and/or polishing steps may be included both upstream and/or downstream of the affinity purification step. Suitable complementary purification steps include size exclusion liquid chromatography, bead-elute liquid chromatography, ionic exchange purification (such as anionic exchange), charged membrane separation, and various other purification and/or polishing strategies used in the art.

In additional aspects, the present invention relates to pharmaceutical compositions comprising the EVs as described herein. Normally, the EVs are produced by an EV-producing cell source which also leads to the incorporation of a DOI into the EVs. The EVs (i.e. the population of EVs) is then typically formulated in a pharmaceutically acceptable composition, which may comprise a pharmaceutically acceptable excipient, carrier and/or diluent or similar. Furthermore, the EVs and/or the pharmaceutical composition may be used in medicine to treat various diseases, disorders, ailments or illnesses. More specifically, the present invention relates to use in the prophylaxis and/or treatment and/or alleviation of a variety of diseases. Non-limiting examples of diseases and conditions include the following non-limiting examples: Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy and other muscular dystrophies, all lysosomal storage diseases such as Gaucher disease type I, II and/or III, Fabry's disease, MPS I, II (Hunter syndrome), and III, Niemann-Pick disease type A, B, and C, Pompe disease, cystinosis, etc., urea cycle disorders such as N-Acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), neurodegenerative diseases including Alzheimer's disease, Parkinson's disease, GBA associated Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the NA cargo molecule in question, or the EV population as such.

The invention and its various aspects, embodiments, alternatives, and variants will now be further exemplified with the enclosed examples, which naturally also may be modified considerably without departing from the scope and the gist of the invention.

### EXAMPLE 1

### Affinity Purification of mRNA-Loaded MSC-Derived Exosomes

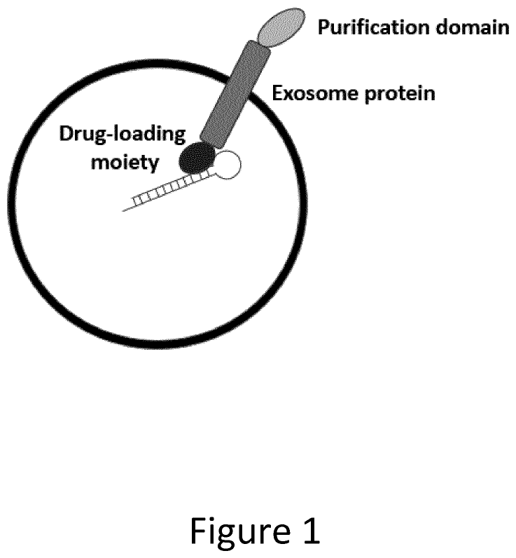

Wharton's jelly-derived MSCs were cultured in conventional tissue culture flasks and transiently transfected using PEI transfection to enable loading and expression of a fusion protein comprising the following domains: the exosome protein CD63, the Z domain (obtained from the staphylococcal Protein A) as the purification domain, and the Cas6 protein as an drug-loading moiety which enables the binding and loading of mRNA into exosomes. The WJ-MSCs were also co-transfected with a construct encoding for an mRNA encoding for nanoluciferase. The engineered EVs are schematically illustrated in FIG. 1.

The EV-containing supernatant from the transfected cells was harvested after 48 hours. The EVs were isolated and purified using two different downstream purification paths for comparative purposes: (1) a combination of tangential flow filtration (TFF) and bead-elute liquid chromatography using the Captocor column (GE Healthcare Life Sciences), and (2) a combination of tangential flow filtration (TFF) and the IgG Sepharose 6 Fast Flow affinity resin (GE Healthcare Life Sciences). Using methodology (1), EV-containing media was collected and subjected to a low speed spin at 300 g for 5 minutes, followed by 2000 g spin for 10 minutes to remove larger particles and cell debris. The supernatant was then filtered with a 0.22 μm syringe filter and subjected to the different purification methodologies. The TFF was carried out using the Vivaflow 50R tangential flow (TFF) device (Sartorius) with 100 kDa cutoff filters or the KR2i TFF system (SpectrumLabs) with 100 or 300 kDa cutoff hollow fibre filters. The preconcentrated medium was subsequently loaded onto the bead-eluate columns (HiTrap Capto Core 700 column, GE Healthcare Life Sciences), connected to an ÄKTAprime plus (GE Healthcare Life Sciences). Flow rate settings for column equilibration, sample loading and column cleaning in place procedure were chosen according to the manufacturer's instructions. Samples were collected according to the UV absorbance chromatogram and concentrated using an Amicon Ultra-15 10 kDa molecular weight cut-off spin-filter (Millipore) to a final volume.

Using methodology (2), the TFF step was carried out as described above, followed by running the EV preparation through the IgG Sepharose 6 Fast Flow affinity resin (GE Healthcare Life Sciences). The cell culture supernatant was loaded onto the IgG Sepharose Fast Flow 6 which was connected to an ÄKTAprime plus. Flow rate settings for the column equilibration, sample loading and column cleaning were chosen according to the manufacturer's instructions. A binding buffer containing 0.05M Tris-HCl, 0.15M NaCl with pH set to 7.6 was used. An elution buffer comprising 0.5M HAc, with a pH of approximately 6 was utilized to elute the IgG-bound EVs comprising the Z domain purification moiety. Competitive elution using isolated Z domain per se was also evaluated separately, with good results. The sample was collected according to the UV absorbance chromatogram and concentrated using an Amicon Ultra-15 10 kDa molecular weight cut-off spin-filter (Millipore) to a final volume of 100 μl and stored at −80° C. for further downstream analysis.

The enrichment of nanoluciferase mRNA in the resultant EV populations was assessed using qPCR. The RNA from 1E10 EVs was extracted using standard methods and retro transcribed using oligo dT to asses the amount of full length RNA molecules. The number of RNA molecules loaded was calculating by absolute quantification using qPCR. The expression of CD63-ZZ-Cas6 was detected both in cells and EVs using standard western blotting. Using downstream purification based on TFF and Captocor, mRNA molecules were present in around 15% of the EVs present in the final population. Using the purification approach which combines TFF and the IgG Sepharose 6 resin, the final population of EVs had nanoluciferase mRNA present in approximately 65% of all EVs, thus resulting in approximately 4-5 fold higher drug enrichment in the final product.

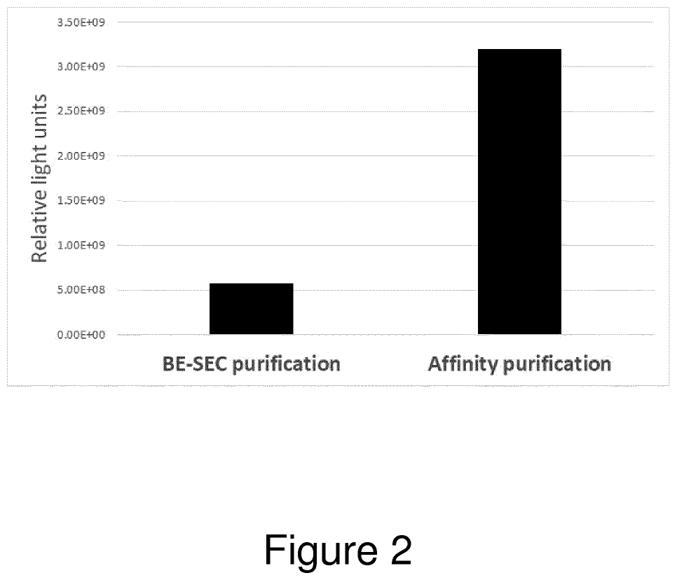

The WJ-MSC exosomes were also evaluated in an in vitro uptake assay. Briefly, Huh7 cells were seeded in cell culture plates followed by exposure for 4 hours to the mRNA loaded engineered exosomes. The bioluminescence generated by the nanoluciferase mRNA was measured by harvesting the cells and measuring total bioluminescence output. The signal from the cells treated with the engineered EVs purified using the IgG Sepharose 6 column was approximately 5 times higher than the signal from the EVs purified using the TFF-Captocor purification approach (FIG. 2).

### EXAMPLE 2

### Affinity Purification of mRNA-Loaded HEKs-Derived Exosomes

Human embryonic kidney cells 293 (HEK293) were stably transduced with a lentiviral system to enable expression of a fusion protein comprising the following domains: the exosomal protein Lamp2B, an hexahistidine (H6) tag as a purification domain in the N-terminal, and a double stranded RNA-binding domain (RBD) from the Tar RNA binding protein 2 (TBPR2) as a drug-loading moiety. A variant fusion protein comprising a self-cleavable intein protein element was also evaluated, with the intein introduced between Lamp2b and the RBD was also evaluated. The HEK293 cells were also transiently co-transfected with a plasmid coding for an shRNA specific for the C-MYC oncogene. The TRBP2 drug-loading domain of the fusion protein enables loading of the shRNA into exosomes, followed by intein-mediated release of the shRNA drug cargo.

The EV-containing supernatant from the transfected cells was harvested 48 hours after plasmid transfection. The EVs were isolated and purified using two different downstream purification paths for comparative purposes: (1) a combination of tangential flow filtration (TFF) and bead-elute liquid chromatography using the Captocor column (GE Healthcare Life Sciences), and (2) a combination of tangential flow filtration (TFF) and the HisTrap HP histidine-tagged protein purification columns (GE Healthcare Life Sciences). Using methodology (1), EV-containing media was collected and subjected to a low speed spin at 300 g for 5 minutes, followed by 2000 g spin for 10 minutes to remove larger particles and cell debris. The supernatant was then filtered with a 0.22 μm syringe filter and subjected to the different purification methodologies. The TFF was carried out using the Vivaflow 50R tangential flow (TFF) device (Sartorius) with 100 kDa cutoff filters or the KR2i TFF system (SpectrumLabs) with 100 or 300 kDa cutoff hollow fibre filters. The preconcentrated medium was subsequently loaded onto the bead-eluate columns (HiTrap Capto Core 700 column, GE Healthcare Life Sciences), connected to an ÄKTAprime plus (GE Healthcare Life Sciences). Flow rate settings for column equilibration, sample loading and column cleaning in place procedure were chosen according to the manufacturer's instructions. Samples were collected according to the UV absorbance chromatogram and concentrated using an Amicon Ultra-15 10 kDa molecular weight cut-off spin-filter (Millipore) to a final volume.

Using methodology (2), the TFF step was carried out as described above, followed by running the EV preparation through the HisTrap HP histidine-tagged protein purification column (GE Healthcare Life Sciences) which are packed with Ni sepharose high performance affinity resin. Essentially, this resin is highly cross-linked with agarose beads with a coupled chelating group (this chelating group is pre-charged with nickel (Ni-NTA)). The column equilibration, sample loading and column cleaning protocols were chosen according to the manufacturer's instructions. The Histidine tag which is present on the outside of the engineered EVs selectively binds with pre-charged Nickel under binding buffer of 20 mM sodium phosphate, 300 mM sodium chloride (in PBS) with 10 mM imidazole (pH 7.4). Column was washed using buffer containing 20-40 mM imidazol in PBS (pH 7.4). His-tagged EVs were then eluted from the HisTrap column using elution buffer containing 300 mM imidazole in PBS (pH 7.4).

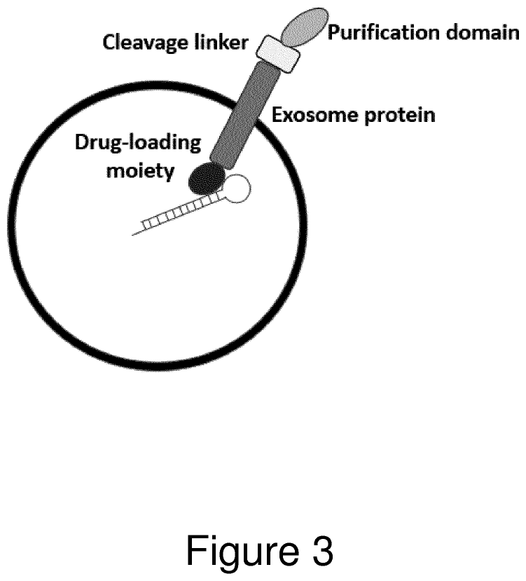

As two alternative approaches, the fusion protein was modified to comprise either a TEV linker peptide or a SUMO linker peptide introduced between the histidine and the exosomal protein LAMP2B (FIG. 3 shows a schematic illustration of the EVs). This allowed us to use a non-imidazol-based elution strategy for the captured EVs, namely a so called targeted proteolytic clipping procedure. Thus, following the Ni-NTA affinity binding of the polyHis-tagged EVs, an elution buffer containing TEV or SUMO protease (concentration determined based on the enzymatic activity) in 0.1-0.5M NaCl (for TEV protease cleavage, along with 40 mM Tris/HCl pH7.5, 2 mM MgCl2, 250 mM sucrose, and for SUMO protease-mediated clevage with 25 mM Tris-HCl (pH 8.0), 0.1% Igepal, 50% (v/v)glycerol) was used, which triggered elution of the captured engineered EVs as a result of proteolytic cleavage.

To quantify the amount of loaded shRNA, the RNA from 1E10 EVs was extracted using standard methods. For absolute quantification of anti-c-myc shRNA in exosomes, a standard curve of a synthetic shRNA was prepared. The amount of c-myc shRNA was normalized either by the number of particles determined by NTA or by the total amout of protein measured by Micro BCA protein assay kit (Thermo Scientific). The expression of Hisx6-Lamp2b-TRBP2 was detected both in cells and EVs using Western blotting. Using downstream purification based on TFF and Captocor, shRNA molecules were present in around 45% of the EVs present in the final population. Using the purification approach which combines TFF with the HisTrap HP purification column, the final population of EVs had c-my shRNA present in approximately 90% of all EVs, thus resulting in approximately 2-fold higher drug enrichment in the final product. Similarly, EVs modified with fusion proteins comprising the drug-loading moiety, the exosomal protein, the SUMO or TEV cleavage linker, and the poly-His showed a similar enrichment of the shRNA in question, however the use of these two constructs enabled enzymatic removal of the purification domain (in this case the His tag).

The engineered HEK293 EVs were also evaluated in an in vitro assay. Briefly, Huh7 cells were seeded in cell culture plates followed by exposure for 4 hours to the shRNA-loaded engineered EVs. The decrease of c-myc RNA was assessed by harvesting the cells and evaluating knockdown using qPCR. The amount of c-myc RNA detected when the cells were treated with the EVs purified using the HisTrap HP purification column was approximately 50% lower than the RNA from cells treated with EVs purified using the TFF-Captocor purification approach (data not shown).

### EXAMPLE 3

### Affinity Purification of sgRNA-Loaded ASCs-Derived Exosomes

Human amniotic epithelial stem cells (hAEs) were cultured in 24 deep well plates with circular cylindrical bottom and transiently transfected using PEI to enable expression of a fusion protein comprising Cas9 (from Streptococcus pyogenes) as a drug-loading protein, the exosomal protein syntenin, a transmembrane gp130 domain to anchor the fusion proteins into the EV membrane, and a maltose-binding protein (MBP) tag. The AE cells were also co-transfected with a plasmid coding for a sgRNA against the IGF2BP1 gene. The co-expression of both plasmids enables the binding of the sgRNA by the Cas9 and loading of the RNA cargo into EVs.

The EV-containing supernatant from the transfected cells was harvested after 48 hours. As in example 1 and 2, the EVs were isolated and purified using two different downstream purification processes ((1) TFF combined with bead-elute LC and (2) TFF followed by the MBPTrap HP affinity resin (GE Healthcare Life Sciences). Using the second method, the TFF step was carried out as described above, followed by running the EV preparation through the MBP Trap high purification column (GE Healthcare Life Sciences) which pre-packed with dextrin sepharose high performance affinity resin. The column equilibration, sample loading and column cleaning were chosen according to the manufacturer's instructions. The MBP tag which is present on the outside of the engineered EVs selectively binds with pre-packed Dextrin sepharasoe under binding buffer of 20 mM Tris-HCl, 200 mM NaCl, 1 mM EDTA (pH 7.4). MBP-tagged EVs were then eluted from the MBP Trap HP column using elution buffer containing 10 mM Maltose. As in example 2, in an alternative fusion protein design, a TEV peptide linker was included to enable enzymatic removal of the MBP tag.

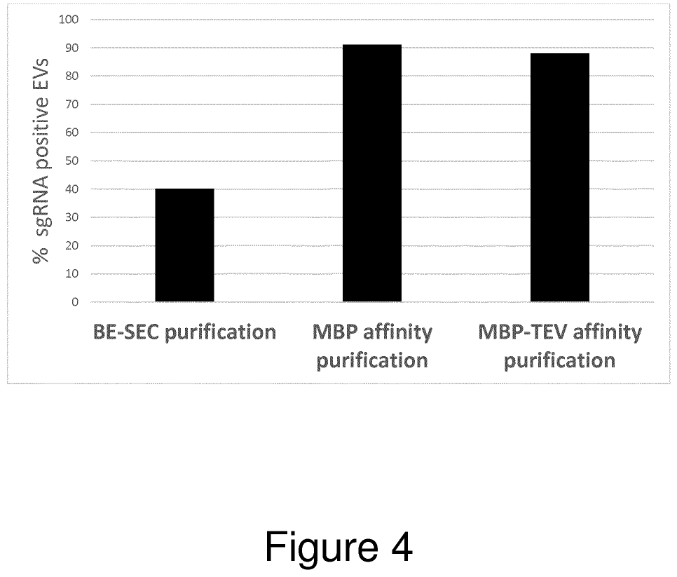

To quantify the amount of loaded sgRNA, the RNA from 1E10 EVs was extracted, followed by quantitation against a standard curve of a synthetic sgRNA. The amount of IGF2BP1 sgRNA was normalized either by the number of particles determined by NTA or by the total amount of protein measured by Micro BCA protein assay kit (Thermo Scientific). The expression of MBP-gpr130-syntenin-Cas9 was detected both in cells and EVs using Western blotting. TFF combined with bead-elute chromatography resulted in sgRNA cargo molecules being present in around 40% of the EVs present in the final population. Using the purification approach which combines TFF and the MBP-based affinity capture (and in the alternative approach subsequent enzymatic removal of the MBP tag), the final population of EVs had IGF2BP1 sgRNA present in approximately 90% of all EVs, thus resulting more than 2-fold higher drug cargo loading in the final EV population (FIG. 4).

1. A fusion protein comprising (i) an EV polypeptide, (ii) a purification domain, and (iii) a drug loading moiety.

2. The fusion protein according to claim 1, wherein the drug-loading moiety is a nucleic acid (NA)-binding protein or a protein-binding domain.

3. The fusion protein according to claim 1, wherein the EV polypeptide is selected from the group comprising CD9, CD53, CD63, CD81, CD82, CD54, CD50, FLOT1, FLOT2, CD49d, CD71, CD133, CD138, CD235a, ALIX, Syntenin-1, Syntenin-2, Lamp2b, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, ARRDC1, LFA-1, LGALS3BP, Mac-1 alpha, Mac-1 beta, MFGE8, SLIT2, STX3, TCRA, TCRB, TCRD, TCRG, VTI1A, VTI1B, and any other EV polypeptides, and any combinations, derivatives, domains, mutated variants, and/or regions thereof.

4. The fusion protein according to claim 1, wherein the EV polypeptide is a transmembrane or membrane-associated polypeptide.

5. The fusion protein according to claim 4, wherein the transmembrane or membrane-associated EV polypeptide is selected from the group comprising CD63, CD81, CD9, CD82, CD44, CD47, CD55, LAMP2B, ICAMs, integrins, and any other EV polypeptides, and any combinations, derivatives, domains, mutated variants or regions thereof.

6. The fusion protein according to claim 1, wherein the EV polypeptide is a non-transmembrane polypeptide fused to a transmembrane or membrane-associated polypeptide which locates the fusion protein to the exosomal membrane.

7. The fusion protein according to claim 6, wherein the non-transmembrane EV polypeptide is fused to a transmembrane polypeptide which locates the fusion protein to the exosomal membrane.

8. The fusion protein according to claim 2, wherein the NA-binding protein is selected from the group comprising mRNA-binding proteins, miRNA-binding proteins, pre-rRNA-binding proteins, tRNA-binding proteins, small nuclear or nucleolar RNA-binding proteins, non-coding RNA-binding proteins, transcription factors, nucleases, RISC proteins, and any combination, derivative, domain or part thereof.

9. The fusion protein according to claim 2, wherein the NA-binding protein is any one of PUF, PUF531, PUFx2, DDX3X, EEF2, EEF1A1, HNRNPK, HNRNPM, HNRNPA2B1, HNRNHPH1, HNRNPD, HNRNPU, HNRNPUL1, NSUN2, Cas6, Cas13, Cas9, WDR1, HSPA8, HSP90AB1, MVP, PCB1, MOCS3, DARS, ELC2, EPRS, GNB2L1, IARS, NCL, RARS, RPL12, RPS18, RPS3, RUVBL1, TUFM, hnRNPA1, hnRNPA2B1, DDX4, ADAD1, DAZL, ELAVL4, ELAVL1, IGF2BP3, HNRNPQ, RBFOX1, RBFOX2, U1A, PPR family, ZRANB2, NUSA, IGF2BP1, IGF2BP2, Lin28, KSRP, SAMD4A, TDP43, FUS, FMR1, FXR1, FXR2, EIF4A1-3, MS2 coat protein, DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and any combination, derivative, domain, region, site, mutated variants or part(s) thereof.

10. The fusion protein according to claim 1, wherein the purification domain is a receptor, an antibody-binding polypeptide, an Fc-binding polypeptide, poly-histidine, glutathione S-transferase (GST), maltose-binding protein (MBP), calmodulin-binding peptide (CBP), intein-chitin binding domain (I-CBD), streptavidin, avidin, FLAG epitope tag, HA epitope tag, T7 tag, S-tag, CLIP, DHFR, cellulose-binding domain, and any combination, derivative, domain or part thereof.

11. The fusion protein according to claim 10, wherein the Fc-binding polypeptide is selected from the group comprising Protein A, Protein G, Protein A/G, Protein L, Protein LG, Z domain, ZZ domain, human FCGRI, human FCGR2A, human FCGR2B, human FCGR2C, human FCGR3A, human FCGR3B, human FCGRB, human FCAMR, human FCERA, human FCAR, mouse FCGRI, mouse FCGRIIB, mouse FCGRIII, mouse FCGRIV, mouse FCGRn, FcIII peptide, and any combination, derivative, domain or part thereof.

12. A complex between a fusion protein according to claim 1 and a drug of interest, wherein the drug-loading moiety of said fusion protein is capable of binding the drug of interest.

13. The complex according to claim 12, wherein the drug of interest is an NA agent, a protein, and/or a peptide.

14. The complex according to claim 13, wherein the NA agent is selected from the group comprising shRNA, miRNA, mRNA, gRNA, sgRNA, pri-miRNA, pre-miRNA, circular RNA, piRNA, tRNA, rRNA, snRNA, lncRNA, antisense oligonucleotide, ribozyme, double-stranded DNA, single-stranded DNA, mini-circle DNA, and/or plasmid DNA.

15. The complex according to claim 13, wherein the NA agent comprises at least one naturally occurring or artificially introduced region or site to which the NA-binding protein binds.

16. The complex according to claim 13, wherein the NA agent encodes for a therapeutic protein.

17. The complex according to claim 16, wherein the therapeutic protein is selected from the group comprising antibodies, antibody fragments, antibody derivatives, single domain antibodies, intrabodies, single chain variable fragments, affibodies, enzymes, transporters, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins, structural proteins, neurotrophic factors, membrane transporters, nucleotide binding proteins, heat shock proteins, CRISPR-associated proteins, and any combination thereof.

18. An extracellular vesicle (EV) comprising a fusion protein according to claim 1.

19-23. (canceled)

24. A polynucleotide encoding for a fusion protein according to claim 1.

25. A cell comprising a polynucleotide according to claim 24.

26-27. (canceled)

28. A method for producing EVs comprising a fusion protein comprising (i) an EV polypeptide, (ii) a purification domain, and (iii) a drug loading moiety, the method comprising the steps of:
(i) introducing into an EV-producing cell a polynucleotide according to claim 24; and,
(ii) allowing for the EV-producing cell to produce EVs comprising the fusion protein.

29. A method for producing EVs comprising a drug of interest, the method comprising the steps of:
(i) introducing into an EV-producing cell a polynucleotide according to claim 24 and a polynucleotide encoding for a drug of interest; and,
(ii) allowing for the EV-producing cell to produce EVs comprising the fusion protein, wherein the drug-loading moiety of said fusion protein binds to the drug of interest and transports it into the EV.

30-31. (canceled)

32. A method of purification of EVs comprising a drug of interest, the method comprising the steps of:
(i) providing an EV according to claim 18;
(ii) allowing the purification moiety of the fusion protein comprised in the EV to bind a purification ligand; and,
(iii) removing EVs that have not bound to the purification ligand.

33-34. (canceled)

35. A pharmaceutical composition comprising the EVs according to claim 18 and a pharmaceutically acceptable carrier.

36. A method of treating a disease comprising administering the pharmaceutical composition according to claim 35.
